(19) United States
(12) Patent Application Publication
(10) Pub. No.: US 2025/0099507 A1
(43) Pub. Date: Mar. 27, 2025

(54) Protein Composition for Repairing Hair Follicle and Preparation Method therefor and Use thereof

(71) Applicant: BEIJING DARWIN BIOTECH CO., LTD., Beijing (CN)

(72) Inventors: Yu WANG, Beijing (CN); Wenyong GAO, Beijing (CN); Lin CHEN, Beijing (CN); Jianjun LI, Beijing (CN)

(73) Assignee: BEIJING DARWIN BIOTECH CO., LTD., Beijing (CN)

(21) Appl. No.: 18/833,708

(22) PCT Filed: Jan. 28, 2023
(86) PCT No.: PCT/CN2023/073590

§ 371 Date: Jul. 26, 2024

(30) Foreign Application Priority Data
Jan. 28, 2022 (CN) 202210105729.X
Apr. 2, 2022 (CN) 202210351455.2
Jul. 5, 2022 (CN) 202210793711.3

(51) Int. Cl. A61K 35/28; A61P 17/14; C12N 5/0775; C12P 21/06

(52) U.S. Cl. CPC A61K 35/28; A61P 17/14; C12N 5/0668; C12P 21/06

(57) ABSTRACT

Provided is a protein composition for repairing hair follicles for repairing hair follicles, wherein the preparation method therefor comprises the following steps: adding any one of or a combination of 20 U/mL-35 U/mL of a nuclease or an omnipotent nuclease to a cell protein extract, performing enzymatic hydrolysis at 37° C.±1° C. for 15-40 minutes, and separating and purifying the prepared enzymatic hydrolysate. The obtained cell protein extract and protein composition have the effects of repairing damaged hair follicle cells, efficiently repairing damaged hair follicles and significantly improving the activity of hair follicle, and have the advantages of a high purity, a good stability, being safe and effective, and effectively solving the problems of living cells needing to be refrigerated, and the activity thereof being limited by cell viability duration, etc.

### TECHNICAL FIELD

The present disclosure belongs to a field of biopharmaceutical technology and specifically relates to a protein composition for repairing hair follicles for repairing hair follicle, preparation method therefor, and use thereof.

### BACKGROUND

Thick hair is an important sign of physical health and hair health. Hair follicle health is the foundation of hair health, and the key to maintaining hair follicle health is to activate the hair follicle matrix cells within the scalp system. Hair follicles undergo a cyclical growth cycle of growth phase, regression phase, and resting phase. The cyclical changes of hair follicles are closely related to the cyclical division, proliferation, and differentiation of hair follicle matrix cells. Highly active hair follicle matrix cells continuously release a large amount of hair follicle matrix cell growth factors, promoting hair follicle cells to enter a virtuous cycle of self-repair, growth, division, and hair growth.

Modern people are affected by factors such as busy life, work pressure, and environmental pollution, leading to problems such as hair breakage, premature aging, severe hair loss, and hair regeneration disorders, which seriously affect their personal image and even cause psychological disorders. According to survey data from the National Health Commission, the number of people with hair loss in China exceeds 260 million, with about 63% being male, mainly suffering from androgenic alopecia. The population with hair loss shows a clear trend of being younger, with 60% of the population experiencing hair loss symptoms around the age of 25. The proportion of hair loss in the post-90s generation is as high as 84%, and the proportion with severe hair loss is as high as 46.7%.

Hair diseases include physiological or pathological manifestations such as hypotrichosis, hypertrichosis, abnormal hair distribution, abnormal hair density, abnormal hair diameter, abnormal hair pigmentation, and abnormal hair shaft in patients. Hair loss includes physiological hair loss and pathological hair loss. Physiological hair loss includes natural hair loss, seasonal hair loss, infant hair loss, senile hair loss, and postpartum hair loss. Pathological alopecia includes both non-scarring alopecia and scarring alopecia. Non-scarring alopecia includes androgenic alopecia (accounting for about 95%), alopecia areata (accounting for about 4%), syphilitic alopecia, trichotillomania, telogen effluvium, anagen effluvium, traction alopecia, mechanical alopecia, folliculitis decalvans, dissecting cellulitis, congenital hypotrichosis, etc. After removing the pathogenic factors of non-scarring hair loss, the hair will regrow. Scarring hair loss will permanently destroy hair follicles, leading to permanent hair loss. The causes include trauma, infection, inflammatory skin diseases (such as discoid lupus erythematosus, hair lichen planus, etc.), scalp lichen planus, scalp discoid lupus erythematosus, radiative hair loss, female fibrous hair loss, scalp burns or scalds, etc.

Patients with pathological hair loss exhibit abnormal hair and excessive shedding which leads to a significant decrease in hair volume. Common causes of hair loss include excessive androgens, genetics, excessive stress, pregnancy, diseases, medications and injuries, etc. Autoimmune diseases, thyroid diseases, lupus erythematosus, diabetes, iron deficiency, eating disorders, anemia and other diseases lead to changes in hormone levels in the body and aggravate hair loss. Antitumor drugs, blood thinners, antihypertensive drugs, contraceptive drugs, burns, various injuries and X-ray radiation, etc. can cause temporary hair loss.

Seborrheic alopecia (Androgenetic Alopecia, AGA) is a hair disease characterized by excessive sebum secretion from the scalp, gradual thinning, softening, and reduction in hair volume, which may be accompanied by symptoms such as scalp itching, dandruff, and pruritus. It is a progressive hair follicle disorder that is influenced by genetic factors and is dependent on androgen action. The total number of AGA patients in China exceeds 100 million, with a male prevalence rate (20.2%) higher than females (5.1%).

Cells are the fundamental unit of life activities and the foundation of body health. When the redox balance of the body is disrupted, it can cause an interruption in redox signaling and control, leading to oxidative stress damage and various diseases. Previous studies have shown that the number and activity of hair follicle stem cells, as well as the regenerative ability of hair follicles, are directly related to hair loss. A decrease in the number and activity of hair follicle stem cells leads to a weakened differentiation ability of hair follicle cells, which affects the hair regeneration cycle and leads to the closure of hair follicles, resulting in permanent hair loss. The main methods for treating hair loss include hair care therapy, medication treatment, laser hair generation and hair transplantation, etc. Hair care therapy mainly uses topical products such as hair growth fluids or agents to stimulate the scalp and promote scalp blood circulation, promoting blood supply, head nutrition, and hair growth in the head. However, there are shortcomings such as poor hair growth and hair loss prevention, and ineffective treatment for damaged and dormant hair follicles. Drug therapy includes oral medications (such as finasteride, spironolactone, VB2, VB6, tanshinone capsules, compound glycyrrhizic acid glycoside tablets, total paeoniflorin capsules, etc.) and topical medications (such as minoxidil solution, Hasinolone solution, selenium disulfide shampoo, etc.), but there are shortcomings such as poor treatment effect, significant side effects, and drug dependence in treating hair loss. Laser hair growth utilizes low-energy laser irradiation with a wavelength of 670 nm to enhance cell activity, accelerate blood microcirculation, increase blood supply and nutrition to the head, promote hair to absorb nutrients, improve the vitality of hair cells and hair follicle stem cells, accelerate protein synthesis, activate hair follicles, etc. However, it has drawbacks such as slow hair growth and high cost. Hair transplantation requires surgery to select hair follicles from the patient's occipital and temporal regions as the source of hair. After separating the collected hair follicles into single or multiple hair follicles, fine microsurgical surgery is performed to transplant the single or multiple hair follicles to the desired hair loss area, allowing the newly transplanted single or multiple hair follicles to survive and grow at the new hair transplant area, thereby repairing the distribution and density of local hair. Although hair transplantation technology can solve the problem of complete closure of hair follicles, it is limited by the transplantation area and hair follicle resources. Additionally, hair transplantation surgery is painful, carries a risk of infection, the survival rate of newly transplanted hair follicles is not guaranteed, it cannot solve the problem of repairing damaged hair follicles, leaves scars on the transplant areas that affect aesthetics, and the cost is high. For this reason, there is an urgent need in clinical practice for a hair growth combination that has better hair growth and anti-hair loss effects, definite therapeutic effects, and safe and effective.

When cells and microorganisms are stimulated by the external stimuli and induced by external stressors (including cold, heat, acid, alkali, current, radiation, chemicals, etc.), they are induced to produce stress proteins. Reference 1 (New limonophyllines A-C from the stem of Atalantia monophylla and cytotoxicity against cholangiocarcinoma and HepG2 cell lines, Arch. Pharm. Res. (2018) 41:431-437) reveals that compounds 1-16 extracted from the Rutaceae plant (Atalantia monophylla) exhibiting activities such as inhibiting tumor cell growth.

Mesenchymal stem cells (MSCs) have the potential for self-replication and multi-lineage differentiation, and they are widely present in tissues such as bone marrow, fat, synovium, dental pulp, amniotic fluid, placenta, umbilical cord, embryo, umbilical cord blood, amniotic membrane, peripheral blood, muscle, urine, etc. They have the characteristics of wide sources, no need for matching, low infection rate, strong differentiation potential, strong proliferation ability, and easy collection. They can produce active factors such as stem cell growth factor (SCF), nerve growth factor (NGF), interleukin-6 (IL-6), interleukin-7 (IL-7), tumor necrosis factor (TNF), interferon (IFN), etc, which involve in regulating cell growth, apoptosis, differentiation, antiviral therapy, immune maturation, and other processes, and are used for immune regulation, tissue repair, and the treatment of acute lung injury, severe pneumonia, acute respiratory distress syndrome, and other diseases. However, MSCs products need to be refrigerated in their production, storage, transportation, and application processes, and their cell viability should be maintained for ≤12 hours, which limits their therapeutic applications. Therefore, it is necessary to develop safe and effective hair follicle repair drugs to meet clinical needs.

### SUMMARY

The purpose of the present disclosure is to provide a protein composition for repairing hair follicles, comprising the following steps:
(1) adding any one of or a combination of 20 U/mL-35 U/mL a nuclease or an omnipotent nuclease to a cell protein extract, and performing enzymatic hydrolysis at 37° C.±1° C. for 15-40 minutes to obtain an enzymatic hydrolysate;
(2) under conditions of 2° C.-8° C., preparing the enzymatic hydrolysate obtained in step (1) to a 5-15 mg/ml solution with an eluent, and then passing through a chromatographic column with an eluent flow rate of 0.1-1 mL/min, monitoring and collecting an eluent fraction with a UV wavelength of 280 nm, wherein the eluent consists of 50 mmol/L phosphate buffer (pH 6.8) containing 300 mmol/L sodium chloride.

In the preferred technical solution of the present disclosure, a method for preparing the protein extract comprises the following steps:
S-1: placing mesenchymal passaged cells with a density of 5.0×10⁶ cells/mL to 1.0×10⁷ cells/mL in a culture medium containing DMEM/F12 40-50%, RPMI1640 40-50%, bovine serum albumin (BSA) 0.1-2%, epidermal growth factor (EGF) 1-15 μg/mL, fibroblast growth factor (FGF) 1-15 μg/mL, insulin transferrin 1-15 μg/mL, compound amino acids (18AA) 0.01-0.1%, and 2-10 μmol/L of a stressor, and then culturing the cells under conditions of 37.0° C.±0.5° C. and 5%±1.0% CO₂ for 10 to 60 minutes, and then separating, washing and collecting the cells, wherein the stressor is selected from any one of compounds 1-16 or a combination thereof;

| Compounds | General formula | Substituents |
|---|---|---|
| 1 | 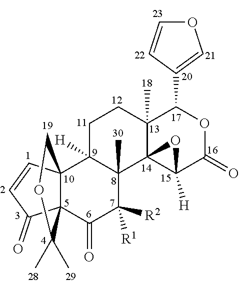 | R¹ = OCH₃, R² = OH |
| 2 | | R¹ = R² = OH |
| 3 | | R¹ = OH, R² = H |
| 4 | 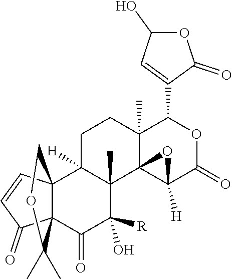 | R = H |
| 5 | | R = OH |
| 6 | 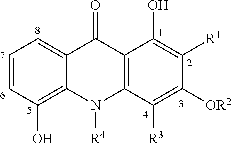 | R¹ = prenyl, R² = H, R³ = prenyl, R⁴ = CH₃ |
| 7 | | R¹ = prenyl, R² = H, R³ = prenyl, R⁴ = H |
| 8 | | R¹ = H, R² = H, R³ = OCH₃, R⁴ = CH₃ |
| 9 | | R¹ = H, R² = CH₃, R³ = OCH₃, R⁴ = CH₃ |
| 10 | | R¹ = OCH₃, R² = H, R³ = H, R⁴ = CH₃ |
| 11 | | R¹ = OCH₃, R² = H, R³ = |
| | | OCH₃, R⁴ = CH₃ |
| 12 | | R¹ = prenyl, R² = CH₃, R³ = H, R⁴ = H |
| 13 | 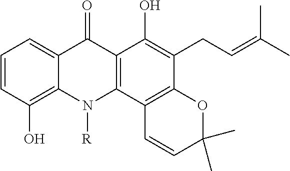 | R = H |
| 14 | | R = CH₃ |
| 15 | 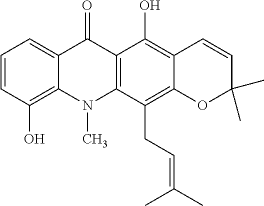 [SMILES: CC(C)=CCc1c2c(c(O)c3c(=O)c4cccc(O)c4n(C)c13)C=CC(C)(C)O2] | |

S-2: dispersing the collected cells in a solvent at a density of 5.0×10⁶ cells/mL-5.0×10⁷ cells/mL, and then obtaining a cell lysate by sonicating at 2° C.-8° C., wherein the solvent is selected from any one or combination of physiological saline, 5% glucose solution, phosphate buffer solution (PBS), TBPS buffer, TBST buffer, Tris buffer;
S-3: separating the cell lysate prepared in step S-2, then sequentially filtering a separated solution through 0.45 μm and 0.22 μm filter membranes, thereby obtaining the cell protein extract.

In the preferred technical solution of the present disclosure, the culture medium in step S-1 contains DMEM/F12 42-45%, RPMI1640 42-45%, bovine serum albumin (BSA) 0.5-1.5%, epidermal growth factor (EGF) 5-10 μg/mL, fibroblast growth factor (FGF) 5-10 μg/mL, insulin transferrin 5-10 μg/mL, compound amino acids (18AA) 0.02-0.05%, and 3-8 μmol/L of stressors.

In the preferred technical solution of the present disclosure, the culture medium in step S-1 contains DMEM/F12 45%, RPMI1640 45%, bovine serum albumin (BSA) 0.5%, epidermal growth factor (EGF) 10 μg/mL, fibroblast growth factor (FGF) 10 μg/mL, insulin transferrin 10 μg/mL, compound amino acids (18AA) 0.05%, and 4-6 μmol/L of stressors.

In the preferred technical solution of the present disclosure, the density of mesenchymal stem cells in step S-1 is 6.0×10⁶-2.0×10⁷ cells/mL, preferably 8.0×10⁶-1.0×10⁷ cells/mL.

In the preferred technical solution of the present disclosure, the mesenchymal stem cells of step S-1 are cultured in a culture medium for 15-50 minutes, preferably 20-40 minutes.

In the preferred technical solution of the present disclosure, the solvent for washing cells in step S-1 is selected from any one or a combination of physiological saline, 5% glucose solution, phosphate buffer (PBS), TBPS buffer, TBST buffer, Tris buffer, with a cell washing frequency of 2-5 times, preferably 3-4 times.

In the preferred technical solution of the present disclosure, the separating described in step S-1 is selected from any one or a combination of centrifugation and filtration, wherein centrifugation conditions are 1000-2000 rpm*3-15 min, preferably 1200 rpm˜1500 rpm*5-10 min.

In the preferred technical solution of the present disclosure, conditions for sonicating in step S-2 are: operating at 2° C.-8° C., 25 KHZ, 360 W for 3 seconds with a gap of 1 second, and sonicating for 1-5 minutes.

In the preferred technical solution of the present disclosure, the separating described in step S-3 is selected from any one or a combination of 2000-8000 rpm*10-30 min centrifugation, multi-stage centrifugation, and multi-stage filtration, preferably 3000-7000 rpm*15-25 min.

In the preferred technical solution of the present disclosure, the multi-stage centrifugation in step S-3 is sequentially 3000-4000 rpm*3-5 minutes, 5000-6000 rpm*3-5 minutes, and 7000 rpm*5-8 minutes.

In the preferred technical solution of the present disclosure, a pore size of the multi-stage filtration membrane is selected from any one of 80 μm, 50 μm, 30 μm, 10 μm, or 5 μm.

In the preferred technical solution of the present disclosure, the cell protein extract prepared in step S-3 is hydrolyzed by either a nuclease or an omnipotent nuclease before separation and purification.

In the preferred technical solution of the present disclosure, the nuclease is selected from any one or a combination of an RNA nuclease and a DNA nuclease.

In the preferred technical solution of the present disclosure, the molecular weight of the protein composition for repairing hair follicles is 20 kDa to 300 kDa, preferably 50 kDa to 200 kDa.

In the preferred technical solution of the present disclosure, the cell protein extract prepared in step S-3 or the protein composition prepared in step (2) is frozen, preferably at −40° C. to −20° C.

In the preferred technical solution of the present disclosure, a freeze-dried protectant is added to the cell protein extract prepared in step S-3 or the protein composition prepared in step (2), and freeze-dried to obtain a cell protein extract freeze-dried preparation or a protein composition freeze-dried preparation, wherein the freeze-dried protectant is selected from any one or a combination of mannitol, sorbitol, dextran, glycerol, sucrose, trehalose, glucose, lactose, maltose, glucan, glycerol trioctanoate (HES), polyethylene glycol, ethylene glycol, phosphates, acetates, citrates, sorbitol or starch.

In the preferred technical solution of the present disclosure, the cell protein extract freeze-dried preparation or the protein composition freeze-dried preparation contains a freeze-dried protectant of 0.5-8%, preferably 1-5%, by mass percentage.

In the preferred technical solution of the present disclosure, a protein stabilizer is optionally added to the cell protein extract prepared in step S-3 or the protein composition prepared in step (2), wherein the protein stabilizer is selected from any one of albumin, a zinc salt, or an aluminum salt.

The preferred technical solution of the present disclosure, wherein the cell protein extract freeze-dried preparation or the protein composition freeze-dried preparation has a pH of 6-8, preferably pH 7-7.5.

In the preferred technical solution of the present disclosure, a protein composition in the protein composition for repairing hair follicles is shown in FIG. 2.

In the preferred technical solution of the present disclosure, the freeze-dried preparation is remelted with an isotonic solution before use, and then used in any or a combination of application, rolling needle, microneedle, massage, intravenous injection, intramuscular injection, subcutaneous injection, acupoint injection, or lumbar puncture, wherein the isotonic solution is selected from any or a combination of physiological saline, 5% glucose solution, phosphate buffer (PBS), TBPS buffer, TBST buffer, or Tris buffer.

In the preferred technical solution of the present disclosure, the cultivation of primary mesenchymal stem cells is a cultivation method in this field.

In the preferred technical solution of the present disclosure, the cultivation of mesenchymal stem cells comprises the following steps: adding primary mesenchymal stem cells to a passaging medium with an initial density of 5.0×10⁵-5.0×10⁶ cells/ml, and then culturing them under conditions of 37.0° C.±0.5° C. and 5%±1.0% CO₂ for 10-15 days. Every 2-3 days, after observing the yellowing of the passaging medium, half of the passaging medium is replaced, wherein the passaging medium contains DMEM/F12 medium containing 10% FBS, 100 U/ml penicillin, and 100 μg/ml streptomycin.

In the preferred technical solution of the present disclosure, the cultivation of primary mesenchymal stem cells comprises the following steps:
A: after cleaning and disinfecting the umbilical cord, dissecting the tissue, taking the Wharton's Jelly tissue, cut it into 3 mm³ small pieces, centrifuge, clean, collect tissue blocks, place them in DMEM/F12 medium containing 10% fetal bovine serum FBS, 100 μg/ml penicillin, and 100 μg/ml streptomycin, and then culture them under conditions of 37.0° C.±0.5° C. and 5%±1.0% CO₂, then changing half of the medium every 2-3 days and culturing until the tissue blocks climb out of the cells;
B: shaking and collecting low-layer cells, washing with PBS, adding 0.25% trypsin and digesting for 2-3 minutes, adding an equal volume of trypsin termination solution to stop digestion, gently blowing with a straw, centrifuging at 1200-1500 rpm/min*5-8 minutes, and collecting the cells.

The purpose of the present disclosure is to provide a method for preparing a cell protein extract with hair follicle repair efficacy, comprising the following steps:
S-1: placing mesenchymal passaged cells with a density of 5.0×10⁶ cells/mL to 1.0×10⁷ cells/mL in a culture medium containing DMEM/F12 40-50%, RPMI1640 40-50%, bovine serum albumin (BSA) 0.1-2%, epidermal growth factor (EGF) 1-15 μg/mL, fibroblast growth factor (FGF) 1-15 μg/mL, insulin transferrin 1-15 μg/mL, compound amino acid (18AA) 0.01-0.1%, and 2-10 μmol/L of a stressor, and then culturing it under conditions of 37.0° C.±0.5° C. and 5%±1.0% CO₂ for 20 to 40 minutes, then separating, washing, and collecting the cells, wherein the stressor is selected from any one of compounds 1-16 or a combination thereof;
S-2: dispersing the collected cells in a solvent at a density of 5.0×10⁶ cells/mL-5.0×10⁷ cells/mL, and then sonicating at 2° C.-8° C. to prepare a cell lysate, wherein the solvent is selected from any one or combination of physiological saline, 5% glucose solution, phosphate buffer solution (PBS), TBPS buffer, TBST buffer, Tris buffer;
S-3: separating the cell lysate prepared in step S-2, filtering the solution obtained through 0.45 μm and 0.22 μm filter membranes in turn to obtain the cell protein extract.

In the preferred technical solution of the present disclosure, the culture medium in step S-1 contains DMEM/F12 42-45%, RPMI1640 42-45%, bovine serum albumin (BSA) 0.5-1.5%, epidermal growth factor (EGF) 5-10 μg/mL, fibroblast growth factor (FGF) 5-10 μg/mL, insulin transferrin 5-10 μg/mL, compound amino acids (18AA) 0.02-0.05%, and 3-8 μmol/L of stressors.

In the preferred technical solution of the present disclosure, the culture medium in step S-1 contains DMEM/F12 45%, RPMI1640 45%, bovine serum albumin (BSA) 0.5%, epidermal growth factor (EGF) 10 μg/mL, fibroblast growth factor (FGF) 10 μg/mL, insulin transferrin 10 g/mL, compound amino acids (18AA) 0.05%, and 4-6 μmol/L of stressors.

In the preferred technical solution of the present disclosure, the density of mesenchymal stem cells in step S-1 is 6.0×10⁶-2.0×10⁷ cells/mL, preferably 8.0×10⁶-1.0×10⁷ cells/mL.

In the preferred technical solution of the present disclosure, the mesenchymal stem cells of step S-1 are cultured in a culture medium for 15-50 minutes, preferably 20-40 minutes.

In the preferred technical solution of the present disclosure, the solvent for washing cells in step S-1 is selected from any one or a combination of physiological saline, 5% glucose solution, phosphate buffer (PBS), TBPS buffer, TBST buffer, Tris buffer, with a cell washing frequency of 2-5 times, preferably 3-4 times.

In the preferred technical solution of the present disclosure, the separation described in step S-1 is selected from any one or a combination of centrifugation and filtration, wherein the centrifugation conditions are 1000-2000 rpm*3-15 min, preferably 1200 rpm˜1500 rpm*5-10 min.

In the preferred technical solution of the present disclosure, the ultrasonic conditions of step S-2 are: operating at 2° C.-8° C., 25 KHZ, 360 W for 3 seconds with a gap of 1 second, and sonicating for 1-5 minutes.

In the preferred technical solution of the present disclosure, the separation described in step S-3 is selected from any one or a combination of 2000-8000 rpm*10-30 min centrifugation, multi-stage centrifugation, and multi-stage filtration, preferably 3000-7000 rpm*15-25 min.

In the preferred technical solution of the present disclosure, the multi-stage centrifugation in step S-3 is sequentially 3000-4000 rpm*3-5 minutes, 5000-6000 rpm*3-5 minutes, and 7000 rpm*5-8 minutes.

In the preferred technical solution of the present disclosure, the pore size of the multi-stage filtration membrane is selected from any one of 80 μm, 50 μm, 30 μm, 10 μm, or 5 μm.

In the preferred technical solution of the present disclosure, the cell protein extract prepared in step S-3 is frozen, preferably at −40° C. to −20° C.

In the preferred technical solution of the present disclosure, the cell protein extract prepared in step S-3 is hydrolyzed by either a nuclease or an omnipotent nuclease before separation and purification.

In the preferred technical solution of the present disclosure, the cultivation of primary mesenchymal stem cells is a cultivation method in this field.

In the preferred technical solution of the present disclosure, the cultivation of mesenchymal stem cells comprises the following steps: adding primary mesenchymal stem cells to a passaging medium with an initial density of 5.0×10⁵-5.0×10⁶ cells/ml, and then culturing them under conditions of 37.0° C.±0.5° C. and 5%±1.0% CO₂ for 10-15 days, every 2-3 days, after observing the yellowing of the passaging medium, half of the passaging medium is replaced, wherein the passaging medium contains DMEM/F12 medium containing 10% FBS, 100 U/ml penicillin, and 100 μg/ml streptomycin.

In the preferred technical solution of the present disclosure, the cultivation of primary mesenchymal stem cells comprises the following steps:
A: After cleaning and disinfecting an umbilical cord, dissecting the tissue, taking the Wharton's Jelly tissue, cutting it into 3 mm³ small pieces, centrifuging, cleaning, collecting tissue blocks, and placing them in DMEM/F12 medium containing 10% fetal bovine serum FBS, 100 μg/ml penicillin, and 100 μg/ml streptomycin, and then culturing them under 37.0° C.±0.5° C. and 5%±1.0% CO₂ conditions, changing half of the medium every 2-3 days until the tissue blocks climb out of the cells;
B: shaking and collecting low-layer cells, washing with PBS, adding 0.25% trypsin for digestion for 2-3 minutes, and adding an equal volume of a trypsin termination solution to stop digestion, gently blowing with a straw, centrifuging at 1200-1500 rpm/min*5-8 minutes, and collecting the cells.

The purpose of the present disclosure is to provide a preparation method for a protein composition for repairing hair follicles, comprising the following steps:
(1) adding any one of or a combination of 20 U/mL-35 U/mL a nuclease or an omnipotent nuclease to a cell protein extract of the present disclosure, and performing an enzymatic hydrolysis at 37° C.±1° C. for 15-40 minutes to obtain an enzymatic hydrolysate;
(2) Under conditions of 2° C.-8° C., preparing the enzymatic hydrolysate obtained in step (1) to a 5-15 mg/ml solution with an eluent, and then passing through a chromatographic column with an eluent flow rate of 0.1-1 ml/min, monitoring and collecting an eluent fraction with a UV wavelength of 280 nm, wherein the eluent consists of 50 mmol/L phosphate buffer (pH 6.8) containing 300 mmol/L sodium chloride.

In the preferred technical solution of the present disclosure, the nuclease is selected from any one or a combination of an RNA nuclease and a DNA nuclease.

In the preferred technical solution of the present disclosure, the molecular weight of the protein composition for repairing hair follicles is 20 kDa to 300 kDa, preferably 50 kDa to 200 kDa.

In the preferred technical solution of the present disclosure, the protein composition obtained in step (2) is frozen, preferably at −4° C. to −20° C.

In the preferred technical solution of the present disclosure, a freeze-dried protectant is added to the protein composition collected in step (2), freeze-dried to obtain a protein composition freeze-dried preparation, wherein the freeze-dried protectant is selected from any one or a combination of mannitol, sorbitol, dextran, glycerol, sucrose, trehalose, glucose, lactose, maltose, glucan, glycerol trioctanoate (HES), polyethylene glycol, ethylene glycol, phosphate, acetate, citrate, sorbitol, starch.

In the preferred technical solution of the present disclosure, the freeze-dried preparation contains a freeze-dried protectant of 0.5-8% by mass percentage, preferably 1-5%.

In the preferred technical solution of the present disclosure, a protein stabilizer is optionally added to the protein composition collected in step (2), wherein the protein stabilizer is selected from any one of albumin, a zinc salt, or an aluminum salt.

The preferred technical solution of the present disclosure is the freeze-dried preparation with a pH of 6-8, preferably a pH of 7-7.5.

In the preferred technical solution of the present disclosure, the protein composition in the protein composition for repairing hair follicles is shown in FIG. 2.

In the preferred technical solution of the present disclosure, the freeze-dried preparation is re-melted with an isotonic solution before use, and then used in any or a combination of application, rolling needle, microneedle, massage, intravenous injection, intramuscular injection, subcutaneous injection, acupoint injection, or lumbar puncture, wherein the isotonic solution is selected from any or a combination of physiological saline, 5% glucose solution, phosphate buffer (PBS), TBPS buffer, TBST buffer, or Tris buffer.

Another object of the present disclosure is to provide a hair follicle repair composition, which consists of any one or a combination of the cell protein extract with hair follicle repair efficacy or the protein composition for repairing hair follicles of the present disclosure and a pharmaceutically acceptable carrier.

The pharmaceutically acceptable dosage or type of carrier of the present disclosure depends on factors such as the physicochemical properties and content of the active ingredients in the composition, formulation type, dissolution and bioavailability of the formulation.

The composition of the present disclosure can be various dosage forms in the field, and can be prepared using formulation techniques in the field.

In the preferred technical scheme of the disclosure, the composition is selected from any kind of freeze-dried preparation, gel, nasal spray, paste, cream, emulsion, liquid dressing, injection and suppository.

In the preferred technical solution of the present disclosure, the administration method of the combination is selected from any one or a combination of application, rolling needle, microneedle, or massage.

Another object of the present disclosure is to provide an application of a cell protein extract with hair follicle repair efficacy, a protein composition for repairing hair follicles, or a combination thereof in preparing products for cell repair and hair follicle repair.

The preferred technical solution of the present disclosure, wherein the product is selected from any one of the following applications: hair growth products, hair disease prevention and treatment products, pathological hair loss prevention and treatment products, delayed physiological hair loss products, and products for improving scalp environment, wherein the hair disease is selected from any one or a combination of hair loss, excessive hair, abnormal distribution, abnormal hair density, abnormal hair diameter, abnormal hair pigment, and hair shaft, the pathological hair loss is selected from androgenic alopecia, alopecia areata, syphilitic alopecia, hair removal fetish, mechanical hair loss, alopecia folliculitis, abscessed penetrating folliculitis, congenital oligotrichosis, and scalp lichen planus. Tinea, lichen planus of hair, discoid lupus erythematosus of the scalp, radiation-induced hair loss, fibrous hair loss in females, or scalp burns. The physiological hair loss is selected from natural hair loss Any of seasonal hair loss, infant hair loss, senile hair loss, or postpartum hair loss.

Another object of the present disclosure is to provide a medication scheme for treating hair loss and preventing hair loss using the hair growth product of the present disclosure. Subjects receive treatment with one or three doses of medication at intervals of two to four weeks as a course of treatment, using either a microneedle or a rolling needle to puncture the hair loss area and apply the medication locally, massaging until fully absorbed.

In the preferred technical solution of the present disclosure, local anesthesia is given to the treatment site before treatment.

Another object of the present disclosure is to provide compounds 1-16 for the application of stress induced stem cell production of functional proteins with repair effects.

In the preferred technical solution of the present disclosure, the repair is any one of a cell repair, a hair follicle repair, a joint repair, or a nerve repair.

Unless otherwise specified, when the present disclosure relates to the percentage between liquids, the said percentage is volume/volume percentage; When the present disclosure relates to the percentage between liquid and solid, the percentage is the volume/weight percentage; When the present disclosure relates to the percentage between solid and liquid, the percentage is the weight/volume percentage; The rest are weight/weight percentage.

Unless otherwise specified, the identification of mesenchymal stem cells (MSCs) in the present disclosure refers to [symbol] Standards for the culture and quality control of umbilical cord mesenchymal stromal cells for neurorestorative clinical application [symbol] .

Compared with prior art, the present disclosure has the following beneficial effects:

1. The present disclosure scientifically screens a culture medium containing stressors to induce mesenchymal stem cells to produce functional proteins with cell repair and hair follicle repair effects, and contains various growth factors and proteins with functions such as regulating cell proliferation and differentiation, cell repair, and cell nutrition. The obtained cell protein extract, protein composition, or their combination have the function of repairing cells and damaged hair follicle cells. After puncturing the hair loss site with any type of microneedle or rolling needle, the protein composition for repairing hair follicles of the present disclosure is locally transdermal introduced, forming an effective drug delivery channel on the scalp surface, directly reaching the hair follicle root, efficiently repairing damaged hair follicles, and significantly improving hair follicle activity. It also has high purity, good stability, safety, and the advantages of effectively solving the problem of living cells requiring refrigeration and their activity being limited by cell viability time, etc, promoting the repair of damaged hair follicles and collagen production, promote hair regeneration, significantly improve accompanying symptoms such as hair itching, excessive oil leakage, dandruff, and patient medication compliance, providing a clinically effective, high absorption rate, non scalp irritant, safe, effective, and simple and fast treatment method.

2. The preparation method of the present disclosure has the advantages of simple operation, green environmental protection, lower cost, and suitability for industrial production.

### BRIEF DESCRIPTION OF THE DRAWINGS

FIG. 1: Electrophoretic separation results of the protein composition for repairing hair follicles of the present disclosure;

FIG. 2 High performance liquid phase detection results of the protein composition for repairing hair follicles of the present disclosure;

FIG. 3: Study on the repair effect of the cell protein extract of the present disclosure on oxidative damaged skin;

FIG. 4: Research results of the experimental group and model group in the study of the hair follicle repair effect of the protein combination for repairing hair follicles of the present disclosure on AGA mouse models;

FIG. 5: H&E staining results of the experimental and model groups in the study of the hair follicle repair effect of the protein combination for repairing hair follicles of the present disclosure on AGA mouse models (scale: 200) u m);

FIG. 6: Quantitative determination of HF in the experimental and model groups of the study on the hair follicle repair effect of the protein combination for repairing hair follicles of the present disclosure on AGA mouse models, where * * * represents p<0.005.

### DETAILED DESCRIPTION OF THE EMBODIMENTS

The following will further explain and describe the detailed content of the present disclosure in conjunction with specific embodiments, but this does not limit the scope of protection of the present disclosure.

1. Culture of primary mesenchymal stem cells

The cultivation of primary mesenchymal stem cells includes the following steps:
1) After cleaning and disinfecting an umbilical cord, dissecting the tissue, taking the Wharton's Jelly tissue, cutting it into 3 mm³ small pieces, centrifuging, cleaning, collecting tissue blocks, placing them in a culture bottle, adding DMEM/F12 culture medium containing 10% fetal bovine serum FBS, 100 μg/ml penicillin, and 100 μg/ml streptomycin, and then culturing it under 37° C. and 5% CO₂ conditions to promote its adhesion. Every 2-3 days, observing the yellowing of the culture medium, replacing half of the culture medium, and culturing for 10-12 days until cells can be seen climbing out at the edge of the tissue blocks;
2) Gently shaking to make the tissue blocks fall off, collecting the tissue blocks and lower-layer cells separately, and then attaching the collected tissue blocks to the wall for culture;
3) After cleaning the collected low-layer cells with PBS, adding an appropriate amount of 0.25% trypsin and digesting for 2-3 minutes, adding an equal volume of trypsin termination solution to stop digestion. Gently blowing the bottom of the bottle with a straw, centrifuging at 1500 rpm/min*5 minutes, and collecting the cells.

2. Passage culture of primary mesenchymal stem cells

Passage culture of primary mesenchymal stem cells: primary mesenchymal stem cells were added to DMEM/F12 medium containing 10% FBS, 100 U/ml penicillin, and 100 μg/ml streptomycin at an initial density of 1.0×10⁵-6.0×10⁵ cells/ml, and then cultured under conditions of 37.0° C.±0.5° C. and 5%±1.0% CO₂ for 10-15 days, with intervals of 2-3 days. After observing the yellowing of the medium, half of the medium was replaced.

3. Reference 1 for the preparation of compounds 1-16 ((New limonophyllines A-C from the stem of Atalantia monophylla and cytotoxicity against cholangiocarcinoma and HepG2 cell lines, Arch. Pharm. Res. (2018) 41:431-437).

### Example 1: Preparation of Cell Protein Extract with Hair Follicle Repair Effect of the Present Disclosure

The preparation method of a cell protein extract with hair follicle repair effect of the present disclosure included the following steps:
(1) adding mesenchymal passaged cells with a density of 5.0×10⁶ cells/mL to the culture medium containing DMEM/F12 45%, RPMI1640 45%, bovine serum albumin (BSA) 0.5%, epidermal growth factor (EGF) 10 μg/mL, fibroblast growth factor (FGF) 10 μg/mL, insulin transferrin 10 μg/mL, compound amino acids (18AA) 0.05% and compound 16 with 5 μmol/L; then, incubating it at 37° C. and 5% CO₂ for 30 minutes, then centrifuging it at 1200 rpm*5 minutes, washing it with PBS three times, and collecting the cells;
(2) dispersing the collected cells in step (1) into physiological saline at a density of 8.0×10⁶ cells/mL, and sonicating for 3 seconds with a gap of 1 second under conditions of 2-8° C., 25 kHz, and 360 W for 2 minutes to prepare a cell lysate;
(3) centrifuging the cell lysate prepared in step (2) at 7000 rpm*20 minutes, then filtering the obtained solution through 0.45 μm and 0.22 μm filter membranes sequentially to obtain the desired solution.

### Example 2: Preparation of the Protein Composition for Repairing Hair Follicles of the Present Disclosure

The preparation of the protein composition for repairing hair follicles of the present disclosure included the following steps:
(1) adding 25 U/mL of UCF. ME UltraNuclease to the cell protein extract prepared in Example 1, and incubating it at 37° C. for 30 minutes to obtain the enzymatic hydrolysate;
(2) under conditions of 2° C.-8° C., preparing the enzymatic hydrolysate prepared in step (1) to a 10 mg/ml solution with an elution, then passing through a high-purity silica gel liquid chromatography protective column (WondaGuard C18, 4.6×5 mm) and a high-purity silica gel liquid chromatography preparation column (SHIMSEN Ankylo C18, 5 μm. 4.6×250 mm), with an elution flow rate of 0.1-1 ml/min, and the elution fraction with a UV wavelength of 280 nm was monitored and collected. The elution consisted of 50 mmol/L phosphate buffer (pH 6.8) containing 300 mmol/L sodium chloride.

### Example 3: Preparation of the Freeze-Dried Preparation of the Protein Composition for Repairing Hair Follicles of the Present Disclosure

Adding the required amount of mannitol to the protein composition prepared in Example 2, stirring, mixing well, and then freeze-drying, and the resulting freeze-dried preparation contained 2% mannitol (m/m).

### Example 4: Preparation of Cell Protein Extract with Hair Follicle Repair Effect of the Present Disclosure

The preparation method of a cell protein extract with hair follicle repair effect of the present disclosure included the following steps:
(1) adding mesenchymal passaged cells with a density of 8.0×10⁶ cells/mL to the culture medium containing DMEM/F12 45%, RPMI1640 45%, bovine serum albumin (BSA) 0.5%, epidermal growth factor (EGF) 10 μg/mL, fibroblast growth factor (FGF) 10 μg/mL, insulin transferrin 10 μg/mL, compound amino acids (18AA) 0.05% and compound 13 with 5 μmol/L; then incubating it at 37° C. and 5% CO₂ for 30 minutes, then centrifuging it at 1200 rpm*5 minutes, washing it with PBS three times, and collecting the cells;
(2) dispersing the collected cells in step (1) into physiological saline at a density of 1.0×10⁷ cells/mL, and sonicating for 3 seconds with a gap of 1 second under conditions of 2-8° C., 25 kHz, and 360 W for 2 minutes to prepare a cell lysate;
(3) centrifuging the cell lysate prepared in step (2) at 7000 rpm*20 minutes, then filtering the obtained solution through 0.45 μm and 0.22 μm filter membranes sequentially to obtain the desired solution.

### Example 5: Preparation of the Protein Composition for Repairing Hair Follicles of the Present Disclosure

The preparation of the protein composition for repairing hair follicles of the present disclosure included the following steps:
(1) adding 20 U/mL of UCF. ME UltraNuclease to the cell protein extract prepared in Example 4, and incubating it at 37° C. for 30 minutes to obtain the enzymatic hydrolysate;
(2) under conditions of 2° C.-8° C., preparing the enzymatic hydrolysate prepared in step (1) with an elution to a 10 mg/ml solution, then passing through a high-purity silica gel liquid chromatography protective column (WondaGuard C18, 4.6×5 mm) and a high-purity silica gel liquid chromatography preparation column (SHIMSEN Ankylo C18, 5 μm. 4.6×250 mm), with an elution flow rate of 0.1-1 ml/min, and the elution fraction with a UV wavelength of 280 nm was monitored and collected. The elution consisted of 50 mmol/L phosphate buffer (pH 6.8) containing 300 mmol/L sodium chloride.

### Example 6: Preparation of the Freeze-Dried Preparation of the Protein Composition for Repairing Hair Follicles of the Present Disclosure

Adding sorbitol to the protein composition prepared in Example 5, stirring well, mixing well, and freeze drying, and the resulting freeze-dried preparation contained 5% sorbitol (m/m).

### Example 7: Preparation of Cell Protein Extract with Hair Follicle Repair Effect of the Present Disclosure

The preparation method of a cell protein extract with hair follicle repair effect of the present disclosure included the following steps:
(1) adding mesenchymal passaged cells with a density of 1.0×10⁷ cells/mL to the culture medium containing DMEM/F12 45%, RPMI1640 45%, bovine serum albumin (BSA) 0.5%, epidermal growth factor (EGF) 10 μg/mL, fibroblast growth factor (FGF) 10 μg/mL, insulin transferrin 10 μg/mL, compound amino acids (18AA) 0.05% and compound 14 with 8 μmol/L; then incubating it at 37° C. and 5% CO₂ for 30 minutes, then centrifuging it at 1200 rpm*5 minutes, washing it with PBS three times, and collecting the cells;
(2) dispersing the collected cells in step (1) into physiological saline at a density of 5.0×10⁷ cells/mL, and sonicating for 3 seconds with a gap of 1 second under conditions of 2-8° C., 25 kHz, and 360 W for 2 minutes to prepare a cell lysate;
(3) centrifuging the cell lysate prepared in step (2) at 7000 rpm*20 minutes, filtering the obtained solution through 0.45 μm and 0.22 μm filter membranes sequentially to obtain the desired solution.

### Example 8: Preparation of the Protein Composition for Repairing Hair Follicles of the Present Disclosure

The preparation of the protein composition for repairing hair follicles of the present disclosure included the following steps:
(1) adding 30 U/mL of UCF. ME UltraNuclease to the cell protein extract prepared in Example 7, and incubating it at 37° C. for 30 minutes to obtain an enzymatic hydrolysate;
(2) under conditions of 2° C.-8° C., preparing the enzymatic hydrolysate prepared in step (1) with an elution to a 10 mg/ml solution, then passing through a high-purity silica gel liquid chromatography protective column (WondaGuard C18, 4.6×5 mm) and a high-purity silica gel liquid chromatography preparation column (SHIMSEN Ankylo C18, 5 μm. 4.6×250 mm), with an elution flow rate of 0.1-1 ml/min, and the elution fraction with a UV wavelength of 280 nm was monitored and collected. The elution consisted of 50 mmol/L phosphate buffer (pH 6.8) containing 300 mmol/L sodium chloride.

### Example 9: Preparation of the Freeze-Dried Preparation of the Protein Composition for Repairing Hair Follicles of the Present Disclosure

Adding mannitol to the protein composition prepared in Example 8, stirring, mixing well, and freeze drying. The resulting freeze-dried preparation contained 5% mannitol (m/m).

### Example 10: Preparation of Cell Protein Extract with Hair Follicle Repair Effect of the Present Disclosure

The preparation method of a cell protein extract with hair follicle repair effect of the present disclosure included the following steps:
(1) adding mesenchymal passaged cells with a density of 5.0×10⁶ cells/mL to a culture medium containing DMEM/F12 45%, RPMI1640 45%, bovine serum albumin (BSA) 0.5%, epidermal growth factor (EGF) 10 μg/mL, fibroblast growth factor (FGF) 10 μg/mL, insulin transferrin 10 μg/mL, compound amino acids (18AA) 0.05% and compound 15 with 2 μmol/L; incubating it at 37° C. and 5% CO₂ for 30 minutes, then centrifuging it at 1200 rpm*5 minutes, washing it with PBS three times, and collecting the cells;
(2) dispersing the collected cells in step (1) into physiological saline at a density of 8.0×10⁶ cells/mL, and sonicating for 3 seconds with a gap of 1 second under conditions of 2-8° C., 25 kHz, and 360 W for 2 minutes to prepare a cell lysate;
(3) centrifuging the cell lysate prepared in step (2) at 7000 rpm*20 minutes, filtering the obtained solution through 0.45 μm and 0.22 μm filter membranes sequentially to obtain the desired solution.

### Example 11: Preparation of the Protein Composition for Repairing Hair Follicles of the Present Disclosure

The preparation of the protein composition for repairing hair follicles of the present disclosure included the following steps:
(1) adding 30 U/mL of UCF. ME UltraNuclease to the cell protein extract prepared in Example 10, and incubating it at 37° C. for 30 minutes to obtain an enzymatic hydrolysate;
(2) under conditions of 2° C.-8° C., preparing the enzymatic hydrolysate prepared in step (1) with an elution to a 10 mg/ml solution, then passing through a high-purity silica gel liquid chromatography protective column (WondaGuard C18, 4.6×5 mm) and a high-purity silica gel liquid chromatography preparation column (SHIMSEN Ankylo C18, 5 μm. 4.6×250 mm), with an elution flow rate of 0.1-1 ml/min, and the elution fraction with a UV wavelength of 280 nm was monitored and collected. The elution consisted of 50 mmol/L phosphate buffer (pH 6.8) containing 300 mmol/L sodium chloride.

### Example 12: Preparation of the Freeze-Dried Preparation of the Protein Composition for Repairing Hair Follicles of the Present Disclosure

Adding dextran to the protein composition prepared in Example 11, stirring well, mixing well, and freeze drying. The resulting freeze-dried preparation contained 1% dextran (m/m).

### Example 13: Preparation of Freeze-Dried Cell Protein Extract Preparation of the Present Disclosure

Adding the required amount of mannitol to the cell protein extract prepared in Example 1, stirring, mixing well, and freeze drying. The resulting freeze-dried preparation contained 2% mannitol (m/m).

### Example 14: Molecular Weight Distribution Detection of the Protein Composition for Repairing Hair Follicles of the Present Disclosure

Using standard molecular weight polyacrylamide gel electrophoresis to detect the molecular weight distribution of the protein composition for repairing hair follicles of the disclosure, included the following steps:

1. Selecting a glass plate with a thickness of 1.5 mm and placing it horizontally, spreading the gel solution (15% lower layer gel, 4% separating gel) prepared according to Table 1 onto the glass plate in sequence, and inserting the comb vertically onto the lower layer gel.

TABLE 1

| reagent | 4% separating gel | | 15% Lower layer gel | |
|---|---|---|---|---|
| ddH₂O | 6 | ml | 3.7 | ml |
| 30% Acrylamide | 1.33 | ml | 8 | ml |
| mixed solution | | | | |
| Tris | 2.5 ml(0.5M Tris pH 6.8) | | 4 ml(1.5M Tris pH 8.8) | |
| 10% SDS | 100 | μL | 160 | μL |
| 10% APS | 100 | μL | 160 | μL |
| TEMED | 10 | μL | 18 | μL |

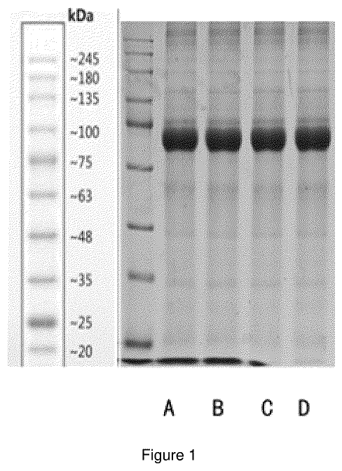

2. After thawing the frozen Marker (20 KDa-245 KDa) stored at −40° C., preparing 10 μg/μL samples of the Marker and the freeze-dried powder of the protein composition for repairing hair follicles of Embodiments 3, 6, 9 and 12 with PBS, adding 20 μL samples to the sample loading hole, performing electrophoresis at 60-80V until clear bands appeared, adjusting the voltage to 100-120V until the Marker was completely separated, placing the PAGE gel in the Coomassie brilliant blue dye solution, and stopped dyeing when clear bands appeared on the gel. After cleaning with pure water, decolorizing with 10% acetic acid solution, and stopped decolorizing when the gel became transparent. The results were shown in FIG. 1, where band A was Example 3, band B was Example 6, band C was Example 9, and band D was Example 12.

### Example 15: High Performance Liquid Chromatography Detection of the Protein Composition for Repairing Hair Follicles of the Present Disclosure

The freeze-dried powder of the protein composition for repairing hair follicles of Example 3 was dissolved in deionized water and to be prepared into a 10 mg/ml test solution.

Chromatographic column: SHIMSEN Ankylo (300 mm*4.6 mm. D., 3 μm; P/N: 380-01215-05) Shimadzu.

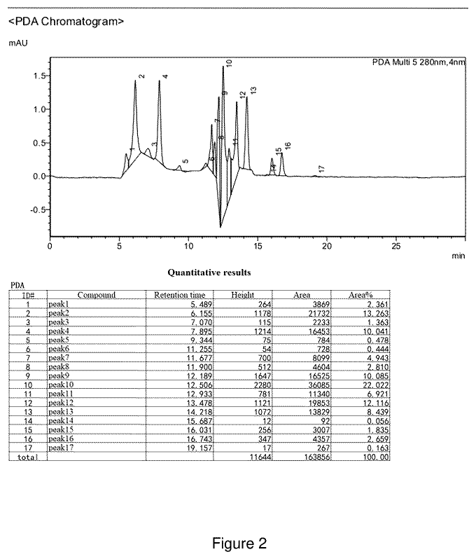

Mobile phase: 50 mmol/L phosphate buffer (pH=6.8), containing 300 mmol/L sodium chloride; Flow rate: 0.3 mL/min; Injection volume: 10 μL; Column temperature: 25° C.; Detection wavelength: 280 nm; Equal elution, collecting for 30 minutes. The results were shown in FIG. 2.

### Experimental Example 1: Study on the Repairing Effect of Cell Protein Extract with Hair Follicle Repair Function on Oxidative Damage in the Present Disclosure

3D skin model: EpiKutis [symbol] , purchased from EpiKutis

SLS working solution: Weighing 0.0080 g of SLS and dissolving it in a 2 mL PBS solution. After filtration at 0.22 μm, a 0.4% SLS mother solution was prepared. And 0.5 mL of PBS was added to 0.5 mL of SLS mother solution, and a 0.2% SLS working solution was prepared.

WY14643 working solution: Weighing 10 mg of WY14643 (PPARα agonist), dissolving it in 1 mL DMSO and preparing a 30 mM WY14643 mother solution. Then adding 10 μL of WY14643 mother solution (30 mM) to a 6 mL of model culture medium and preparing it into a 50 UM of WY14643 working fluid.

Experimental sample: The freeze-dried cell protein extract powder prepared in Example 13 was dissolved in physiological saline to obtain a 1% cell protein extract freeze-dried preparation solution.

Model culture medium: DMEM basic culture medium.

Adding 0.9 mL of model culture medium to the 6-well plate, transferring the 3D skin model to the 6-well plate, and labeling the test number.

Blank control (BC): The skin model was left untreated and incubated in a CO₂ incubator (37° C., 5% CO₂) for 24 hours;

Negative control (NC): Adding 25 μL of 0.2% SLS working solution to the surface of the skin model, incubating it in a CO₂ incubator (37° C., 5% CO₂) for 24 hours;

Positive control (PC): Adding 25 μL of 0.2% SLS working solution to the surface of the skin model and incubating it in a CO₂ incubator (37° C., 5% CO₂) for 24 hours; then adding 25 μL of 50 μM WY14643 working solution and incubating it in a CO₂ incubator (37° C., 5% CO₂) for 24 hours;

Experimental group: Adding 25 μL of 0.2% SLS working solution to the surface of the skin model and incubating it in a CO₂ incubator (37° C., 5% CO₂) for 24 hours; then adding 25 μL of 1% cell protein extract solution, and incubating it in a CO₂ incubator (37° C., 5% CO₂) for 24 hours.

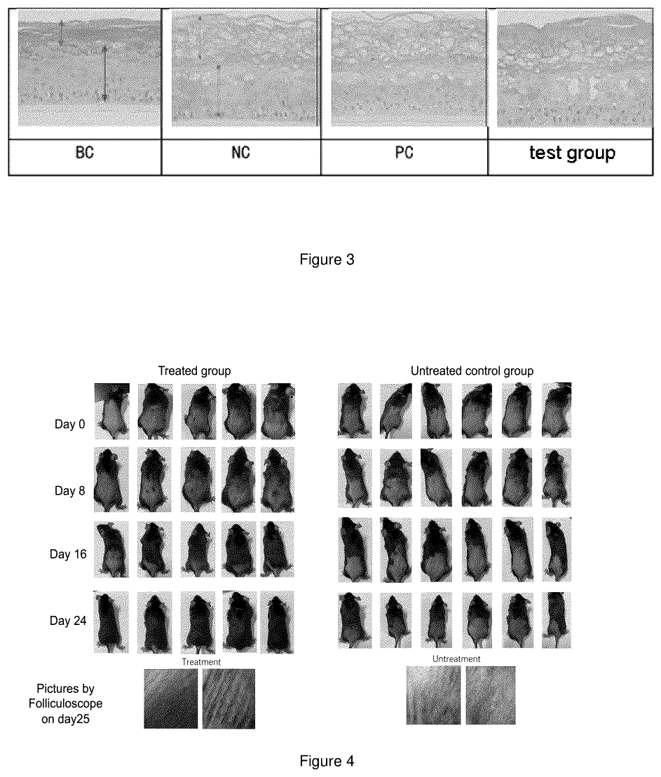

After 24 hours of incubation, washing and removing residual liquid inside and outside of the skin model with PBS solution. After 24 hours of fixation with 4% paraformaldehyde, loop cutting the model and observing after H&E staining. The results were shown in FIG. 3. The cell protein extract of the present disclosure significantly repaired the oxidative damage to the skin.

### Experimental Example 2: Investigation of the Hair Growth Effect of the Protein Composition for Repairing Hair Follicles of the Disclosure

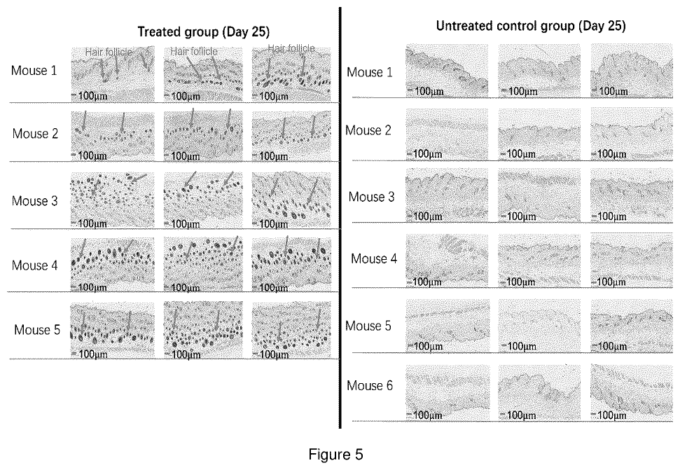

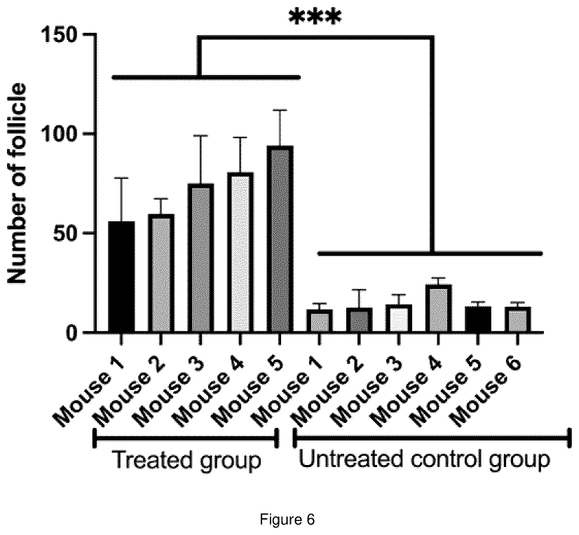

Selecting and weighing 11 C57BL/6J male mice of 20-25 g and randomly dividing them into a model group and a drug group, with 5 mice in the model group and 6 mice in the drug group. AGA mouse models were established by daily local application of testosterone solution to the Experimental animals. First, the back skin of the experimental mice was shaved and removed with animal hair removal cream. Then, 0.5% testosterone ethanol solution (w/v) (prepared by dissolving an appropriate amount of testosterone in 50% ethanol solution (v/v)) was applied to the depilated skin of the experimental mice at a dose of 0.1 mL/cm² per day, and the testosterone ethanol solution was continuously applied to the depilated skin of the experimental mice for 33 days after depilation. Starting from the 8th day after hair removal, the experimental group was applied the hair follicle repair protein combination freeze-dried powder (2 mg dissolved in 200 μl of PBS) of Example 3 of the present disclosure every two days to each mouse at a dose of 2 mg/time, until the 24th day after hair removal. The hair growth of mice on the 0th, 8th, 16th and 24th days after hair removal of experimental animals was observed. On the 25th day after administration of the experimental animal, the mice were euthanized and their back skin was removed for HE staining (scale: 200 μm), Observing the hair growth and hair follicle regeneration of depilated skin, and randomly selecting three fields of view to calculate the number of HFs. The results were shown in FIGS. 4 to 6. The depilated area of the model group mice remained pink, with almost no new hair growth or hair follicle regeneration. More than half of the HFs were in a resting period, and the miniaturized HFs were completely located in the dermis, with incomplete hair tissue and skin morphology. The hair removal area of the experimental group mice showed significant new hair and significantly more regenerated hair follicles, with the formation of enlarged corms and inner root sheaths located in subcutaneous tissue. A large proportion of HFs had entered the growth phase, and by the 33rd day after hair removal, thick hair had grown on the back skin of the mice. The hair tissue and skin morphology were intact, promoting hair follicle growth, maintaining skin morphology, and maintaining skin physiological functions. The protein composition for repairing hair follicles of the present disclosure significantly promoted damaged hair follicle repair, hair regeneration, and activated HF stem cells (p<0.05).

The above description of the specific embodiments of the present disclosure does not limit the present disclosure. Those skilled in the art may make various changes or deformations based on the present disclosure, as long as they do not deviate from the spirit of the present disclosure, they should fall within the scope of protection of the claims of the present disclosure.

1. A protein composition for repairing hair follicles, comprising the following steps:
(1) adding any one of or a combination of 20 U/mL-35 U/mL a nuclease or an omnipotent nuclease to a cell protein extract and performing enzymatic hydrolysis at 37° C.±1° C. for 15-40 minutes to obtain an enzymatic hydrolysate;
(2) under conditions of 2° C.-8° C., preparing the enzymatic hydrolysate obtained in step (1) to a 5-15 mg/ml solution with an eluent, and then passing through a chromatographic column with an eluent flow rate of 0.1-1 mL/min, monitoring and collecting an eluate fraction with a UV wavelength of 280 nm, wherein the eluent consists of 50 mmol/L phosphate buffer (pH 6.8) containing 300 mmol/L sodium chloride.

2. The protein composition as claimed in claim 1, wherein a method for preparing the cell protein extract comprises the following steps:
S-1: placing mesenchymal passaged cells with a density of 5.0×10⁶ cells/mL to 1.0×10⁷ cells/mL in a culture medium containing DMEM/F12 40-50%, RPMI1640 40-50%, bovine serum albumin (BSA) 0.1-2%, epidermal growth factor (EGF) 1-15 μg/mL, fibroblast growth factor (FGF) 1-15 μg/mL, insulin transferrin 1-15 μg/mL, compound amino acids (18AA) 0.01-0.1%, and 2-10 μmol/L of a stressor, and then culturing the cells under conditions of 37.0° C.±0.5° C. and 5%±1.0% CO₂ for 10 to 60 minutes, and then isolating washing, and collecting the cells, wherein the stressor is selected from any one of compounds 1-16 or a combination thereof;

| Compounds | General formula | Substituents |
|---|---|---|
| 1 | 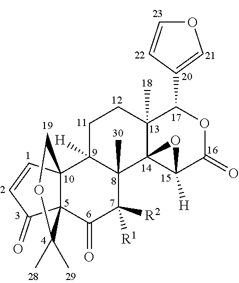 | R¹ = OCH₃, R² = OH |
| 2 | | R¹ = R² = OH |
| 3 | | R¹ = OH, R² = H |
| 4 | 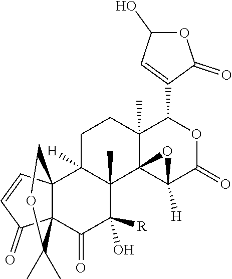 | R = H |
| 5 | | R = OH |
| 6 | 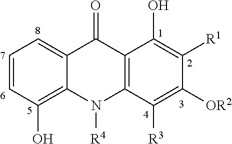 | R¹ = prenyl, R² = H, R³ = prenyl, R⁴ = CH₃ |
| 7 | | R¹ = prenyl, R² = H, R³ = prenyl, R⁴ = H |
| 8 | | R¹ = H, R² = H, R³ = OCH₃, R⁴ = CH₃ |
| 9 | | R¹ = H, R² = CH₃, R³ = OCH₃, R⁴ = CH₃ |
| 10 | | R¹ = OCH₃, R² = H, R³ = H, R⁴ = CH₃ |
| 11 | | R¹ = OCH₃, R² = H, R³ = |
| | | OCH₃, R⁴ = CH₃ |
| 12 | | R¹ = prenyl, R² = CH₃, R³ = H, R⁴ = H |
| 13 | 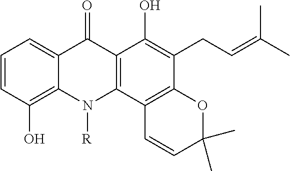 | R = H |
| 14 | | R = CH₃ |
| 15 | 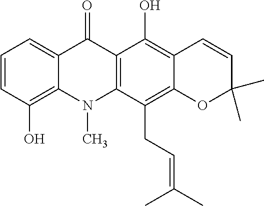 [SMILES: CC(C)=CCc1c2c(c(O)c3c(=O)c4cccc(O)c4n(C)c13)C=CC(C)(C)O2] | |

S-2: dispersing the collected cells in a solvent at a density of 5.0×10⁶ cells/mL-5.0×10⁷ cells/mL, and then obtaining a cell lysate by sonicating at 2° C.-8° C., wherein the solvent is selected from any one or a combination of physiological saline, 5% glucose solution, phosphate buffer solution (PBS), TBPS buffer, TBST buffer or Tris buffer;
S-3: separating the cell lysate prepared in step S-2, then sequentially filtering a separated solution through 0.45 μm and 0.22 μm filter membranes, thereby obtaining the cell protein extract.

3. The protein composition as claimed in claim 1, wherein a molecular weight of the protein composition for repairing hair follicles is 20 kDa to 300 kDa, preferably 50 kDa to 200 kDa.

4. The protein composition as claimed in claim 1, wherein culturing the mesenchymal passage cells of step S-1 in a culture medium for 15-50 minutes, preferably 20-40 minutes.

5. The protein composition as claimed in claim 1, conditions for sonicating of step S-2 are: operating at 2° C.-8° C., 25 kHZ, 360 W for 3 seconds with a gap of 1 second, and sonicating for 1-5 minutes.

6. The protein composition as claimed in claim 1, wherein adding a freeze-dried protectant to the protein composition prepared in step (2), freeze-drying and obtaining a freeze-dried preparation of the protein composition, wherein the freeze-dried protectant is selected from any one or a combination of mannitol, sorbitol, dextran, glycerol, sucrose, trehalose, glucose, lactose, maltose, glucan, glycerol trioctanoate, polyethylene glycol, phosphates, acetates, citrates, or starch.

7. The protein composition as claimed in claim 1, wherein the freeze-dried preparation comprises a freeze-dried protectant of 0.5-8% by a mass percentage, preferably 1-5%.

8. The protein composition as claimed in claim 1, wherein the freeze-dried preparation of the protein composition has a pH of 6-8, preferably a pH of 7-7.5.

9. A hair follicle repair composition comprising a protein composition for repairing hair follicles as claimed in claim 1 and a pharmaceutically acceptable carrier.

10. Use of the protein composition for repairing hair follicles as claimed in claim 1 in the preparation of products for cell repair or hair follicle repair.
